(19) United States
(12) Patent Application Publication
(10) Pub. No.: US 2023/0301925 A1
(43) Pub. Date: Sep. 28, 2023

(54) PHOSPHOLIPID PRESENTING PARTICLES FOR CELL TARGETING IN THERAPY AND DIAGNOSTICS

(71) Applicant: UNIVERSITY OF SOUTH CAROLINA, COLUMBIA, SC (US)

(72) Inventors: KIDOCHUKWU J. ATUBE, COLUMBIA, SC (US); R. MICHAEL GOWER, COLUMBIA, SC (US)

(21) Appl. No.: 18/188,154

(22) Filed: Mar. 22, 2023

Related U.S. Application Data
(60) Provisional application No. 63/322,346, filed on Mar. 22, 2022.

(51) Int. Cl. A61K 9/48; A61K 9/50; A61K 31/519

(52) U.S. Cl. CPC A61K 9/4866; A61K 9/5015; A61K 9/5089; A61K 31/519

(57) ABSTRACT

The present disclosure is generally directed to methods for forming a polymeric particle according to an emulsification/solvent extraction methodology. The method includes combining an aqueous phase with an organic phase to form an emulsion that includes droplets of the organic phase dispersed in the aqueous phase. The aqueous phase may include a first emulsifier. The organic phase includes a second emulsifier and a biocompatible polymer dissolved in a solvent. The method includes removing at least a portion of the solvent from the organic phase upon which the biocompatible polymer solidifies to form a polymeric particle. The second emulsifier is present at a surface of the solidified polymeric particle.

### CROSS REFERENCE TO RELATED APPLICATION

This application claims filing benefit of U.S. Provisional Patent Application Ser. No. 63/322,346, entitled “Phospholipid Presenting Particles for Cell Targeting in Therapy and Diagnostics,” having a filing date of Mar. 22, 2022, which is incorporated herein by reference for all purposes.

### BACKGROUND

Macrophages are immune cells that play essential roles in every stage of tissue regeneration. These immune cells orchestrate tissue regeneration, in part, by playing both sides of the immunological coin. Early inflammatory macrophages predominately infiltrate affected sites and clear cellular debris from damaged tissue. The clearance of cellular debris leads to an immunoregulatory macrophage phenotype that resolves inflammation and promotes regeneration by releasing regenerative and anti-inflammatory factors. Unfortunately, age-related defects, diseases, and massive trauma could lead to prolonged inflammatory responses characterized by a failed resolution of inflammation by macrophages. A failed transition of macrophages from an inflammatory state to an anti-inflammatory and regenerative state impairs the tissue regeneration process. Fortunately, several small molecules that bind intracellular macrophage targets and promote an anti-inflammatory response in the presence of inflammatory stimuli have been identified. However, their receptors are ubiquitously expressed. Thus, these promising therapeutics suffer from on-target, but off-site effects that limit therapeutic efficacy. Hence, a growing need exists to target these small molecules to macrophages for optimal therapeutic efficacy and systemic safety.

Biocompatible and biodegradable polymers can be made into particle drug delivery carriers with different sizes and morphology, particles that can encapsulate both hydrophilic and hydrophobic drugs and also particles with controlled drug release rate. Fabrication of polymeric particle drug delivery carriers is often achieved with the established emulsion/solvent extraction technique. The versatility and scalability of this technique makes it possible to easily fabricate particle drug delivery carriers of varying physical characteristics at a commercial scale. Even more, FDA approval of 19 drug formulations made from poly(lactic-co-glycolic acid (PLG) has made this polymer promising in terms of clinical translation. Thus, making PLG particles favorable drug delivery carriers that measure up to major drug delivery standards and emerge as one of the most investigated polymeric drug delivery carriers at the laboratory and in pharmaceuticals. Unfortunately, the ability to control the surface characteristics of the polymeric particles has not been completely successful. Such issues may result in unintended consequences on the particles formed and affect successful ability to deliver drugs to the intended target.

What are needed in the art are methods for forming polymeric particles that can be utilized to modify the surface reactivity of polymeric particles with ligands for purposes such as intracellular drug delivery.

### SUMMARY

In general, the present disclosure is directed to methods for forming a polymeric particle according to an emulsification/solvent extraction methodology. The method may include combining an aqueous phase with an organic phase to form an emulsion that includes droplets of the organic phase dispersed in the aqueous phase. The aqueous phase may include a first emulsifier. The organic phase may include a second emulsifier and a biocompatible polymer dissolved in a solvent. The method may include removing at least a portion of the solvent from the organic phase upon which the biocompatible polymer solidifies to form a polymeric particle. The second emulsifier may be present at a surface of the solidified polymeric particle.

Also, the present disclosure, in general, is directed to methods for forming a biodegradable drug delivery particle according to an emulsification/solvent extraction methodology. For instance, the method may include combining an aqueous phase with an organic phase to form an emulsion that includes droplets of the organic phase dispersed in the aqueous phase. The aqueous phase may include a first emulsifier. The organic phase may include a second emulsifier, a biocompatible polymer, a biologically active agent, and a solvent. The method may include removing at least a portion of the solvent from the organic phase upon which the biocompatible polymer solidifies to form a polymeric particle. The second emulsifier may be present at a surface of the solidified polymeric particle.

These and other features and aspects, embodiments and advantages of the present invention will become better understood with reference to the following description and appended claims.

### BRIEF DESCRIPTION OF THE DRAWINGS

A full and enabling disclosure of the present disclosure is set forth more particularly in the remainder of the specification, including reference to the accompanying figures, in which:

FIG. 1 illustrates the fabrication of PS:PLG particles.

FIG. 2A depicts particles produced with varying amounts of PS.

FIG. 2B depicts various particle characteristics including mass, particle size, particle size coefficient of variation (CV), and mass yields of particles.

FIG. 2C depicts fluorescence images of Annexin V-PE binding to PS:PLG particles.

FIG. 2D depicts median fluorescence intensity (MFI) of PS:PLG particles analyzed by flow cytometry.

FIG. 2E depicts histogram of fluorescence intensity of each microparticle formulations.

FIG. 2F depicts fluorescence image of Annexin V-PE binding to 1:50 PS:PLG particles incubated in culture media for 48 hrs prior to assay.

FIG. 3A depicts RAW 264.7 macrophage viability with the CCK8 assay after treatment with particles for 24 hours.

FIG. 3B depicts RAW 264.7 macrophage numbers using a hemocytometer following treatment with varying doses of 1:50 PS:PLG particles for 24 hours.

FIG. 3C depicts assessment of membrane integrity with propidium iodide following a 24 hour treatment with 1:50 PS:PLG particles at a dose of 25 particles per cell seeded.

FIG. 4A depicts fluorescence images of macrophage interaction with PLG-C6 particles and 1:50 PS:PLG-C6 particles.

FIG. 4B depicts fluorescence intensity due to C6 particle interaction with RAW 246.7 macrophages.

FIG. 5A depicts TNF-α secreted by RAW 264.7 macrophages treated with either PLG or 1:50 PS:PLG particles at a dose of 10 particles per cell seeded for 24 hrs.

FIG. 5B depicts TGF-β1 secreted by RAW 264.7 macrophages treated with either PLG or 1:50 PS:PLG particles at a dose of 10 particles per cell seeded for 24 hrs.

FIG. 5C depicts TNF-α secreted by RAW 264.7 macrophages co-treated with LPS (100 ng/mL) and either PLG or 1:50 PS:PLG particles at varying doses for 24 hrs.

FIG. 5D depicts IL-10 secreted by RAW 264.7 macrophages co-treated with LPS (100 ng/mL) and either PLG or 1:50 PS:PLG particles at varying doses for 24 hrs.

FIG. 5E depicts TGF-β1 secreted by RAW 264.7 macrophages co-treated with LPS (100 ng/mL) and either PLG or 1:50 PS:PLG particles at varying doses for 24 hrs.

FIG. 6A depicts macrophage interaction with PLG-C6 particles and 1:50 PS:PLG-C6 particles after 6 hrs of incubation.

FIG. 6B depicts bivariate plots of macrophages treated with PLG-C6 particles.

FIG. 6C depicts bivariate plots of macrophages treated with 1:50 PS:PLG-C6 particles.

FIG. 6D depicts percentage of F4/80-positive cells uptake of C6-labeled particles.

FIG. 6E depicts median fluorescent intensity (MFI) of C6-labeled particles internalized by F4/80-positive cells.

FIG. 6F depicts macrophage interaction with 1:50 PS:PLG particles after 6 hrs of incubation.

FIG. 6G depicts macrophage interaction with 1:200 PS:PLG particles after 6 hrs of incubation.

FIG. 6H depicts fluorescent intensity of 1:50 PS:PLG and 1:200 PS:PLG particles.

FIG. 7A depicts particle internalization by BMDMs 6 hrs after particle delivery.

FIG. 7B depicts frequency of particles internalization.

FIG. 8A depicts TNF-α secreted by BMDMs treated with either PLG or 1:50 PS:PLG particles at a dose of 10 particles per cell seeded for 24 hrs.

FIG. 8B depicts IL-10 secreted by BMDMs treated with either PLG or 1:50 PS:PLG particles at a dose of 10 particles per cell seeded for 24 hrs.

FIG. 8C depicts TGF-β1 secreted by BMDMs treated with either PLG or 1:50 PS:PLG particles at a dose of 10 particles per cell seeded for 24 hrs.

FIG. 8D depicts TNF-α secreted by BMDMs co-treated with LPS (100 ng/mL) and either PLG or 1:50 PS:PLG particles at varying doses for 24 hrs.

FIG. 8E depicts IL-10 secreted by BMDMs co-treated with LPS (100 ng/mL) and either PLG or 1:50 PS:PLG particles at varying doses for 24 hrs.

FIG. 8F depicts TGF-β1 secreted by BMDMs co-treated with LPS (100 ng/mL) and either PLG or 1:50 PS:PLG particles at varying doses for 24 hrs.

FIG. 8G depicts IL-6 secreted by BMDMs co-treated with LPS (100 ng/mL) and either PLG or 1:50 PS:PLG particles at varying doses for 24 hrs.

FIG. 9 depicts IL-10 secreted by BMDMs co-treated with LPS (1 ng/mL) and various doses of PS:PLG for 24 hrs.

FIG. 10 depicts TNF-α secreted by BMDMs pre-treated with UNC2025 followed by a co-treatment with LPS (1 ng/mL) and PS:PLG (1:10 cell to particle ratio).

FIG. 11 depicts IL-6 secreted by BMDMs pre-treated with UNC2025 followed by a co-treatment with LPS (1 ng/mL) and PS:PLG (1:10 cell to particle ratio).

FIG. 12 depicts IL-10 secreted by BMDMs pre-treated with UNC2025 followed by a co-treatment with LPS (1 ng/mL) and PS:PLG (1:10 cell to particle ratio).

FIG. 13 depicts TNF-α levels of a mice treated intraperitoneally with either saline or PS-PLG (2 mg).

FIG. 14 depicts IL-6 levels of a mice treated intraperitoneally with either saline or PS-PLG (2 mg).

Repeat use of reference characters in the present specification and drawings is intended to represent the same or analogous features or elements of the present invention.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

The present disclosure is generally directed to methods for forming polymeric particles with a functionalized surface. Incorporating phospholipids into the emulsion results in presentation of the chemical moiety on the surface of the particle. Advantageously, incorporating phospholipids onto the surface of polymeric particles may improve cell internalization of the particle and binding of the particle to mucus. Methods disclosed herein may be utilized to modify the surface reactivity of polymeric particles with phospholipids with applications in intracellular drug delivery and delivery to mucosal membranes.

Forming a surface-functionalized polymeric particle may be utilized as particle drug delivery carriers with tunable morphologies and sizes. Further, the surface-functionalized polymeric particles may encapsulate both hydrophilic and hydrophobic drugs with controlled drug release rates. As utilized herein, “morphology” generally refers to the overall shape and size of a particle, e.g., large, small, spherical, ovoid, cubic, etc. As utilized herein, “topology” generally refers to the presence or lack of surface deformation of a particle, e.g., the existence of features across the surface that lead to additional surface area such as ridges, bumps, dips, etc. or alternatively, a smooth surface.

Through surface functionalization of polymer particles, a variety of particle characteristics may be controlled. For instance, the particle surface may be tuned using functionalization. Tuning particle surface chemistry may impact particle-cell interactions. For instance, in one embodiment, disclosed particles can be designed for use as drug depots and utilized in delivery of a biologically active agent (e.g., a drug) to a delivery site (e.g., a targeted or systemic in vivo delivery site). Through control and modification of the morphology and topology of polymeric particles carrying the agent, release characteristics as well as cell and tissue interactions can be modified and controlled. For instance, a higher surface area particle can have a higher loading of a biologically active agent as compared to a particle having the same average diameter or volume but having a smooth surface. This difference can be particularly large when the biologically active agent is carried at the particle surface. Moreover, interactivity of a particle with particular tissues or cells can vary depending upon the particle's topology. As such, improved delivery to a particular location or particular structures can be attained through control and modification of the particle topography. For instance, particular cells or tissues can exhibit higher interactivity with a particle having a rough surface as compared to a smooth surface. In such an embodiment, formation of a drug delivery depot in the form of particles with a rough surface can increase interaction between the particles and the desired delivery site, which can provide increased drug delivery to the desired site and less off-target delivery. In other embodiments, it may be preferred to form particles having a smooth surface, e.g., to minimize interfacial free energy. Control of the morphology of the particles in combination with control of the topology of the particles can provide additional benefits with regard to loading levels, interaction characteristics, etc.

Particles formed according to disclosed methods are not limited to use in biological applications as drug delivery particles. Improvements, as can be attained through the controlled formation techniques disclosed herein, are applicable to a wide variety of applications, including, without limitation, textiles, coatings applications, filler applications, etc., as particles having well-defined and controlled morphologies and topologies and add-on levels of carried agents can provide improved characteristics such as, and without limitation to hydrophobicity, oleophobicity, rheology, thixotropy, adhesion, etc.

Particles disclosed herein are formed based on oil-in-water emulsion techniques in which an aqueous phase is combined with an organic phase to form an emulsion including droplets of the organic phase dispersed in the aqueous phase. In a standard oil-in-water emulsion technique, the organic phase (e.g., oil phase) includes a polymer dissolved in an organic solvent. The aqueous phase includes water and an emulsifier that partitions itself at the oil/water interface of the dispersed droplets of the organic phase, thereby maintaining the particle droplets throughout emulsification and solidification and preventing agglomeration of the droplets as well as the solidified particles. Following emulsion formation, solvent is removed from the organic droplets upon which the polymer carried in the organic phase solidifies to form the polymeric particles suspended in the aqueous phase.

Methods disclosed herein differ from standard oil-in-water techniques through the addition of a second emulsifier at the surface of polymeric particles. These additions provide a system that allows for modulation of the surface roughness and other characteristics (e.g., add-in level of additives, particle size) of the solidified polymer particles. Without wishing to be bound to any particular theory, it is believed that the second solvent can preferentially carry the second emulsifier to the water/oil interface during solidification, leading to the accumulation of the second emulsifier at the interface where the first and second emulsifiers compete with one another. A localized decrease in surface tension at the interface due to the presence of the less effective emulsifier (which is generally the second emulsifier) causes the droplet to expand irregularly across the interface, which, in turn, leads to folding and deforming of the interface that does not reverse prior to polymer hardening. A high enough concentration of the less effective emulsifier can result in a polymer particle with a non-smooth, rough, or ruffled surface.

The aqueous phase includes water and a first emulsifier that is soluble in water. The first emulsifier may include, but is not limited to, polyvinyl alcohol (PVA), hydroxyethyl cellulose, carboxymethyl cellulose, methyl cellulose, gelatin, alkylarylsulfonates, alkylsulphates, fatty acid salts of alkali metals, polyethylene glycol (PEG), poly(ethylene-alt-maleic acid) (PEMA), didodecyldimethylammonium bromide (DMAB), or a combination thereof. In one embodiment, the first emulsifier is PVA. The first emulsifier may be present in the aqueous phase in an amount from about 0.1 wt. % to about 3 wt. %, such as from about 0.2 wt. % to about 2.5 wt. %, such as from about 0.5 wt. % to about 2 wt. %, such as from about 0.75 wt. % to about 1.5 wt. %, or any range therebetween.

When utilizing a polymeric emulsifier, there are no particular limitations on the type of a polymeric emulsifier that can be utilized. For instance, a polyvinyl alcohol emulsifier can have about 85% or greater hydrolysis of the acetate groups, or about 95% or greater hydrolysis of the acetate groups, or can be fully hydrolyzed in some embodiments. In addition, the size of a polymeric emulsifier is not limited. For instance, the weight average molecular weight of a polymeric emulsifier may be about 5,000 Daltons (Da) or greater, such as from about 5,000 Da to about 500,000 Da, such as from about 10,000 Da to about 200,000 Da, such as from about 20,000 Da to about 50,000 Da, or any range therebetween.

The organic phase includes an organic solvent and a second emulsifier that is understood to compete with the first emulsifier at the oil/water interface during emulsification and solidification. Accordingly, the second emulsifier can be an amphiphilic compound that exhibits surface activity in the emulsion/condensation system. The effectiveness of the two emulsifiers can differ from one another. More specifically, the surface tension at a water/oil interface in the presence of the second emulsifier (and absent the first emulsifier) can differ from the surface tension at a similar oil/water interface in the presence of the first emulsifier (absent the second emulsifier). As such, when both emulsifiers are present, local variations in surface tension can be established across the water/oil interface surfaces of the dispersed oil droplets in disclosed systems, which is believed to lead to the ability to establish topological variations and ‘roughening’ of the surface of solidified particles as described herein.

In some embodiments, the organic phase of a system can include one or more organic solvents. In an embodiment, the second emulsifier may exhibit a higher solubility in one of the solvents of the organic phase as compared to another solvent of the organic phase. This can provide for preferential transport of the second emulsifier to the water/oil interface of dispersed droplets where topological modification can occur.

The second emulsifier may be a small molecule amphiphilic compound. For instance, a second emulsifier may have a number average molecular weight of about 900 g/mol or less, such as about 500 g/mol or less, or such as about 300 g/mol or less in some embodiments. For example, a second emulsifier may have a number average molecular weight of from about 100 g/mol to about 400 g/mol, such as from about 150 g/mol to about 350 g/mol, such as from about 200 g/mol to about 300 g/mol, or any range therebetween.

In one embodiment, a second emulsifier may be a small molecule amphiphilic phenolic compound that includes one or more phenolic groups and is amphiphilic at the emulsion formation conditions. In some embodiments, a second emulsifier may be a polyphenolic compound comprising at least two phenol groups joined by a C1-C6 alkyl chain that can optionally include additional functionality (e.g., additional hydroxyl groups, oxygen, unsaturation, etc.).

In one embodiment, the second emulsifier may include a functionalized phospholipid. For instance, the functionalized phospholipid may include, but is not limited to, phosphatidylserine (PS), phosphatidylethanolamine, phosphatidylcholine, phosphatidylinositol, or a combination thereof. For instance, the second emulsifier may include a phosphatidylserine.

While several ligands have been attached to particle drug delivery carriers, phosphatidylserine remains one of the most utilized in targeting macrophages because of the on the surface of apoptotic cells via a plethora of PS receptors. PS is a phospholipid that remains stationed in the inner plasma membrane of healthy cells but becomes exposed to the outer plasma membrane of apoptotic cells. While on the outer plasma membrane, PS functions as an “eat me” signal to macrophages, thus attracting macrophages to bind and subsequently engulf the expressing apoptotic cell. Interestingly, receptor binding of PS leads to the internalization of apoptotic cells and initiation of an anti-inflammatory and regenerative gene program by macrophages. The ability of PS to not only act as an “eat me” signal but possibly modulate macrophage function in a positive way has made the utility of this phospholipid as a targeting moiety of growing interest. For example, in several studies, liposomal-based drug delivery system have been supplemented with PS to target macrophages and eventually modulate macrophage function in various disease conditions. In one study, PS was presented on the surface of liposome-coated gold nanocages which improved cellular uptake by bone-marrow derived macrophages. Elegantly, these studies either show improved targeting of macrophages with their drug delivery carriers being dependent on PS or improved therapeutic effects of drugs loaded in their drug delivery carriers facilitated by efficient particle uptake by macrophages via PS. Perhaps, owing to the ease of phospholipid incorporation onto liposomes, a prevalent utilization of PS as a macrophage targeting moiety involves liposomal surface presentation. However, liposomal particle technology while refined, present with certain limitations in liposomal stability, uncontrolled drug release profile, challenges in encapsulating hydrophobic small molecules and off-target effects of treatments due to leakiness. Thus, prompting the need to explore other widely accepted drug delivery carriers. The particle performed have enhanced cellular uptake. Particle interaction with macrophages modifies inflammatory responses. This effect can be further modified by small molecule delivery.

In combination with the second emulsifier, the organic phase may include a polymer dissolved in the organic solvent. The polymer of the organic phase may include any polyester homo- or copolymer generally known in typical emulsion condensation particle formation processes. Polyesters can be amorphous, crystalline, and/or a combination thereof. For instance, any polyester including branched or unbranched, and optionally, including chemical unsaturation that is soluble in a water-immiscible organic solvent is encompassed. A polyester can have any glass transition temperature as long as it is soluble in the first solvent at the formation conditions and can have any weight average molecular weight. In some embodiments, the weight average molecular weight can be from about 5,000 Da to about 200,000 Da, such as from about 20,000 Da to about 150,000 Da, such as from about 50,000 Da to about 100,000 Da, or any range therebetween.

Polyesters can be condensation products of unsaturated polybasic acids or of corresponding acid equivalent derivatives thereof including esters, anhydrides or acid chlorides and polyhydric alcohols. Optionally, one or more additional polyacids common in the art of polycondensation may be used in addition to the unsaturated polyacid. Examples of ethylenically unsaturated polyacids include, but are not limited to maleic, fumaric, itaconic, phenylenediacrylic, citraconic and mesaconic acid. Diacids as may be incorporated in a polyester can include, but are not limited to, malonic, succinic, glutaric, adipic, pimelic, azelaic, and sebacic acids, phthalic, isophthalic, terephthalic, tetrachlorophthalic, tetrahydrophthalic, trimellitic, trimesic, isomers of naphthalenedicarboxylic acid, chlorendic acid, trimellitic acid, trimesic acid, and pyromellitic acid. Polyesters can incorporate any of a wide variety of polyhydric alcohols, which are well known in the art of polycondensation and may be aliphatic, alicyclic, or aralkyl. Exemplary alcohols can include, but are not limited to ethylene glycol, 1,3 propylene glycol, 1,6-hexanediol, 1,10 decanediol, 1,4-cyclohexanedimethanol, 1,4 cyclohexanediol, hydroquinone bis (hydroxyethyl) ether, diethylene glycol, neopentyl glycol, bisphenols such as bisphenol A, ethylene oxide and propylene oxide adducts of bisphenol A, pentaerythritol, trimethylolpropane, and polyester polyols, such as that obtained by the ring-opening polymerization of ε-caprolactone. Additionally; A-B type poly-condensation monomers, which contain both hydroxyl and acid derivative functions, can be used, as well as monoacids and monoalcohols.

In some embodiments, particles can be intended for use in biological systems and as such the polyesters used in forming the particles can be biocompatible and, in some embodiments, biodegradable. Biocompatible and biodegradable polyesters as may be utilized can include, without limitation, poly(ethylene oxide)-poly(propylene oxide)-poly(ethylene oxide) (PEO-PPO-PEO) copolymer, poly(lactide-co-glycolide) (PLG) copolymer, polycaprolactone (PCL), poly(lactic acid) (PLA), poly(glycolic acid) (PGA), polyethylene glycol, and polysorbate, as well as any combination of biocompatible, biodegradable polymers.

The weight ratio of the second emulsifier to polymer in the organic phase can be from about 1:5 to about 1:500, such as from about 1:10 to about 1:200, such as from about 1:50 to about 1:100, or any range therebetween. For instance, the weight ratio of the second emulsifier to polymer in the organic phase can be 1:10. In another embodiment, the weight ratio of the second emulsifier to polymer in the organic phase can be 1:50. In yet another embodiment, the weight ratio of the second emulsifier to polymer in the organic phase can be 1:100. In another embodiment, the weight ratio of the second emulsifier to polymer in the organic phase can be 1:200.

The functionalized phospholipid can bind PLG at a surface of the polymeric particle. Surprisingly, PS-presenting PLG particles disclosed herein are not only well tolerated by macrophages, but promoted macrophage-particle interaction and particle internalization. Macrophages are specialized immune cells that are ubiquitous throughout the human body. These immune cells play vital roles in disease states and human health through their profound capacity in initiating innate immune response. However, the incredible functional diversity of macrophages goes beyond defense. Macrophages help shape the normal physiological and anatomical functions of major organs and tissue of the body and help orchestrate every crucial stage of tissue regeneration: homeostasis, inflammation, proliferation and regeneration.

The second emulsifier and biocompatible polymer of the organic phase can be co-dissolved in one or more solvents. The solvent may be an organic solvent that is soluble for components in the organic phase and slightly soluble or insoluble in the aqueous phase and also non-reactive with other components of the system. Solvents may be aliphatic, aromatic, aromatic-aliphatic, saturated or unsaturated, halogenated solvents, an ether, or combinations thereof. Examples of suitable organic solvents may include, without limitation, toluene, xylene, dichloromethane, chloroform, trichloroethylene, tetrachloroethylene, tetrachloroethanes, chlorobenzene, dichlorobenzenes, ethyl acetate, butyl acetate, ethyl formate, methylethyl ketone, and mixtures of these compounds. In some embodiments, it may be preferred to utilize an organic solvent with relatively low toxicity that has been deemed non-hazardous for use in formation of biocompatible systems. For instance, 1,4-dioxane, dichloromethane, chloroform, dimethylformamide, dimethylacetamide or mixtures thereof may be utilized as a solvent in an application intended for biological uses. In one embodiment, the solvent can include dichloromethane.

A system can include additional components. For instance, a system can include detectable labels (e.g., a fluorescent dye or the like). In one embodiment, a component (e.g., a polymer, an emulsifier, etc.) of the system can be modified to carry a detectable label. Alternatively, a detectable label can be added to the polymeric matrix following solidification of the polymer. In one embodiment as described further herein, a component of the system (e.g., a polymeric first emulsifier such as polyvinyl alcohol) can be modified to carry a detectable label that can then be incorporated into the particles in conjunction with the emulsifier. A detectable label can encompass an optically detectable label, e.g., a fluorescent or phosphorescent label. For instance, isothiocyanate-containing labeling agents such as fluorescein isothiocyanate (FITC), tetramethylrhodamine isothiocyanate, or rhodamine B isothiocyanate (RITC) as are readily available in the market can be utilized. Detectable labels are not limited to such materials however. Optically detectable labels can exhibit a relatively long emission lifetime and a relatively large Stokes shift. One type of fluorescent compound that has both a relatively long emission lifetime and relatively large Stokes shift are lanthanide chelates, such as chelates a samarium dysprosium (Dy(III)), europium (EU(III)), and terbium (Tb(III)). Another type of fluorescent compound that has both a relatively long emission lifetime and relatively large Stokes shift are transition metal chelates, such as chelates of ruthenium (Ru(ii)), osmium (Os(II)), and rhenium (Re(I)). These, as well as other detectable labels as are known in the art are encompassed herein.

Other components that can provide a desired functionality to either the formation system or the formed particles can be included. By way of example, biologically active agents can be incorporated in a system that, upon particle formation, can be contained in the particles in a drug delivery application. Exemplary biologically active agents as may be incorporated in a polymeric particle can include, without limitation, small molecules, proteins, carbohydrates, lipids, glycosides, indoles, peptides, polyphenols, nucleic acids, glycans, glycoproteins, glycosaminoglycans, lipoproteins, and the like. In one embodiment, the biologically active agent may be a tyrosine kinase (e.g., FMS-like tyrosine kinase 3 (Flt3) receptor and/or Mer receptor tyrosine kinase) inhibitor. For instance, the tyrosine kinase inhibitor may be UNC2025. The biologically active agent may be delivered intracellularly at a dosage of from about 1 nM to about 1000 nM, such as from about 5 nM to about 800 nM, such as from about 10 nM to about 500 nM, such as from about 50 nM to about 300 nM, such as from about 100 nM to about 200 nM, or any range therebetween.

Particles of any suitable size can be formed, with preferred size generally depending upon the desired application of the particles. For instance, when forming particles for use as a drug depot for in vivo delivery, limiting particle size to a range greater than about 5 microns can inhibit phagocytic clearance, and produce drug release profiles with a relatively long duration. However, smaller particles can likewise be formed by control of the relative concentrations of the polymer and functionalized phospholipid.

Polymeric particles formed based on methods disclosed herein may have a size of from about 100 nm to about 20 μm, such as from about 500 nm to about 15 μm, such as from about 1 μm to about 12 μm, such as from about 5 μm to about 10 μm, or any range therebetween.

Disclosed herein are methods directed to a facile and robust approach of surface-functionalizing PLG particles with phosphatidylserine (PS) using the single oil-in-water emulsion/solvent extraction technique. Varying amounts of functional PS can be presented on the surface of the PLG particles based on the concentration of PS present in the organic phase. These bioinspired PS-presenting PLG particles can be well tolerated by macrophages and can significantly promote macrophage-particle interaction and particle internalization. Disclosed particles may aid in developing drug delivery therapies capable of actively getting drugs or bioactive agents to macrophages for better clinical outcomes as well as in optimizing existing drug release formulations made from PLG, such as the FDA approved Lupron Depot that delivers macrophages.

The preceding description is exemplary in nature and is not intended to limit the scope, applicability or configuration of the disclosure in any way. Various changes to the described embodiments may be made in the function and arrangement of the elements described herein without departing from the scope of the disclosure.

Unless defined otherwise, all technical and scientific terms used herein have the same meaning as commonly understood by one of ordinary skill in the art to which this invention is related.

As used in this application and in the claims, the singular forms “a”, “an”, and “the” include the plural forms unless the context clearly dictates otherwise. Additionally, the term “includes” means “comprises”. The methods and compositions of the present disclosure, including components thereof, can comprise, consist of, or consist essentially of the essential elements and limitations of the embodiments described herein, as well as any additional or optional ingredients, components or limitations described herein or otherwise useful in biocidal compositions.

Unless otherwise indicated, all numbers expressing quantities of ingredients, properties such as molecular weight, percentages, and so forth, as used in the specification or claims are to be understood as being modified by the term “about”. Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited.

As used herein, “optional” or “optionally” means that the subsequently described material, event or circumstance may or may not be present or occur, and that the description includes instances where the material, event or circumstance is present or occurs and instances in which it does not. As used herein, “w/w %” and “wt %” mean by weight as relative to another component or a percentage of the total weight in the composition.

The term “about” is intended to mean approximately, in the region of, roughly, or around. When the term “about” is used in conjunction with a numerical range, it modifies that range by extending the boundaries above and below the numerical values set forth. Unless otherwise indicated, it should be understood that the numerical parameters set forth in the following specification and attached claims are approximations. At the very least, and not as an attempt to limit the application of the doctrine of equivalents to the scope of the claims, numerical parameters should be read in light of the number of reported significant digits and the application of ordinary rounding techniques.

The phrase “effective amount” means an amount of a compound that promotes, improves, stimulates, or encourages a response to the particular condition or disorder or the particular symptom of the condition or disorder.

As used herein, the terms “first”, “second”, and “third” may be used interchangeably to distinguish one component from another and are not intended to signify location or importance of the individual components.

Here and throughout the specification and claims, range limitations are combined and interchanged, such ranges are identified and include all the sub-ranges contained therein unless context or language indicates otherwise. For example, all ranges disclosed herein are inclusive of the endpoints, and the endpoints are independently combinable with each other.

This written description uses examples to disclose the present disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

Furthermore, certain aspects of the present disclosure may be better understood according to the following examples, which are intended to be non-limiting and exemplary in nature. Moreover, it will be understood that the compositions described in the examples may be substantially free of any substance not expressly described.

### EXAMPLES

### Materials and Methods

50:50 Poly (D, L-lactide-co-glycolide) (PLG) with an ester end group and an inherent viscosity of 0.55 dL/g was purchased from Evonik. Porcine brain L-α-phosphatidylserine (PS) was purchased from Avanti Polar Lipids, Inc. Annexin V-PE, Annexin V binding buffer, propidium iodide solution, and the following antibodies: Trustain fcX (anti-CD16/32) and APC anti-mouse F4/80 antibody were all purchased from BioLegend. Cell counting kit 8 (CCK8), lipopolysaccharide (LPS), chloroform, dichloromethane (DCM), poly (vinyl alcohol) (PVA) (MW 13,000-23,000, 87-89% hydrolyzed), coumarin 6 (C6), dimethyl sulfoxide (DMSO), were purchased from Sigma Aldrich. ELISA kits used in this study were all obtained from R&D systems. Dulbecco's Modified Eagle's Medium (DMEM) with 4.5 g/L glucose and L-glutamine and DMEM/F12 was purchased from Corning Cellgro. Recombinant macrophage colony stimulating factor (MCSF) was purchased from Cell Guidance Systems. Hoechst 3342 solution was purchased from ThermoFisher Scientific. Ultrapure water was obtained from a Thermo Scientific Barnstead Nanopure system.

Lyophilized particles were reconstituted in water at a concentration of 0.25 mg/mL. 400 μL of particles were added to a well of a 48-well plate and allowed to settle. Three phase contrast images per well were acquired with an EVOS®FL microscope at 20× magnification. The Particle Analysis plugin in ImageJ was used to measure particle size. At least 7000 particles were analyzed.

Particle mass yield may be calculated according to Equation (1):

Mass Yield (%)=(M_{PT}/M_{PLG}+M_{PS})*100 (1),

where M_{PT} is the mass of particles recovered from the emulsion, M_{PLG} is the mass of PLG added to the emulsion, and M_{PS} is the mass of phosphatidylserine added to the emulsion.

Lyophilized particles were reconstituted in Annexin V binding buffer at 1 mg/mL. Then, 100 μL of particles were incubated with 5 μL of phycoerythrin labeled Annexin V (Annexin V-PE) for 15 mins at room temperature. To remove unbound Annexin V-PE, particles were collected via centrifugation (250×g for 10 min) and washed in Annexin V binding buffer three times. Visualization of Annexin V-PE binding was carried out using an EVOS® FLoid microscope using the RFP light cube (531/40 nm Excitation, 593/40 nm Emission). Quantification of Annexin V binding was carried out using a FACS Aria flow cytometer equipped with a 488 nm laser and optical filters for PE. Flow cytometry data was analyzed using FlowJo software.

In one embodiment, lyophilized particles were reconstituted in DMEM supplemented with 1 mM sodium pyruvate, 1500 mg/mL sodium bicarbonate, 1% penicillin-streptomycin, and 10% FBS and incubated at 37° C. and 5% CO₂ for 48 hours. The particles were then collected by centrifugation and subjected to an Annexin V binding assay. These particles were then analyzed for Annexin V-PE binding with an EVOS® FL microscope.

RAW 264.7 macrophages were obtained from ATCC (Manassas, VA, USA). Cells were maintained at 37° C. and 5% CO₂ in complete media of Dulbecco's modified eagle's medium (DMEM) supplemented with 1 mM sodium pyruvate, 1500 mg/mL sodium bicarbonate, 1% penicillin-streptomycin, and 10% fetal bovine serum (FBS). RAW 264.7 macrophages were expanded in a T25 flask and passaging took place 48 hrs after seeding or when cultures were 60-70% confluent. For particle experiments, RAW 264.7 macrophages were seeded in well plates at 25,000/cm².

Bone marrow-derived macrophages (BMDMs) were derived from the hindlimbs of female Institute of Cancer Research (ICR) mice. After euthanasia, hindlimbs were collected and placed into polypropylene tubes containing ice-cold, sterile 2% FBS in PBS. Bone marrow suspensions were then flushed from bone shafts of femurs and tibias. Suspensions were then passed through a 21 g needle followed by a 70 μm strainer. After centrifuging the suspension at 300×g for 5 mins at 4° C., red blood cells were lysed, and bone marrow-derived cells were counted and seeded into non-tissue culture treated petri dishes at 500,000 cells/cm². Cells were maintained in DMEM/F12 media containing 10% FBS, 1% penicillin-streptomycin, and 10 ng/mL macrophage colony stimulating factor (M-CSF). After 5 days of differentiation, non-adherent cells were removed by gentle washing. Adherent cells were harvested using trypsin with 0.25% EDTA for 5 minutes at 37° C. Flow cytometry indicated that 96% of these cells co-expressed CD45, F4/80, and CD11b, indicating successful macrophage differentiation. For particle experiments, BMDMs were plated into tissue-culture treated well plates at a seeding density of 50,000 cells/cm².

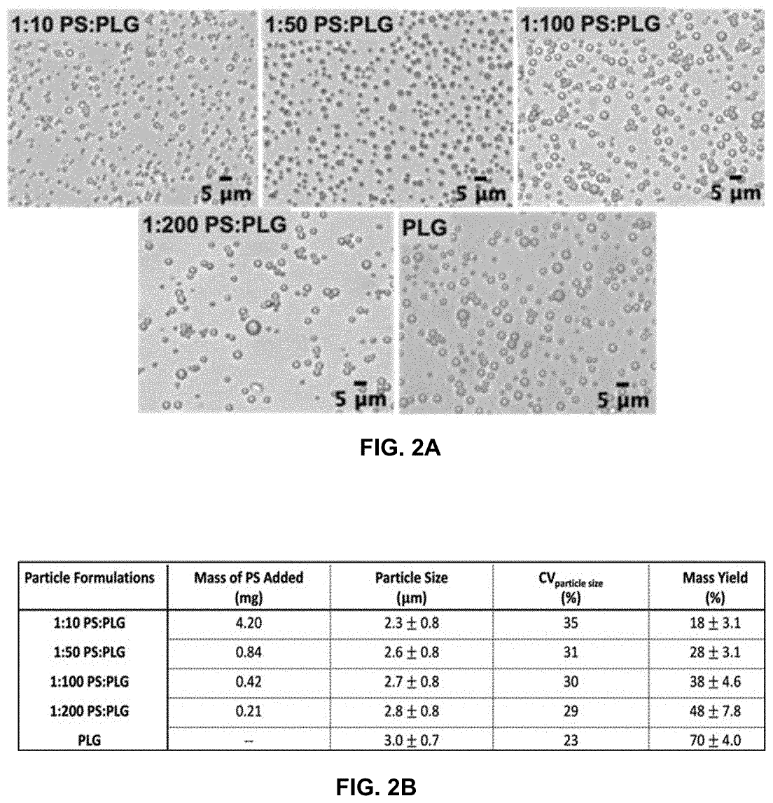

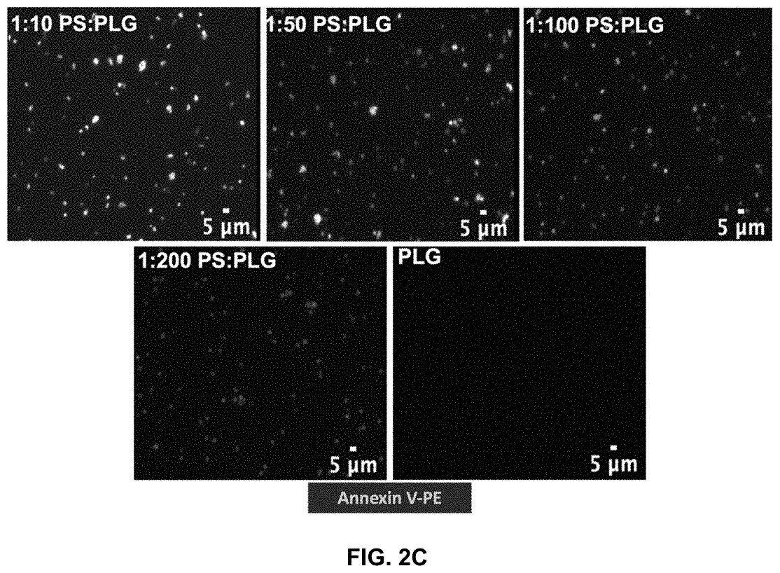

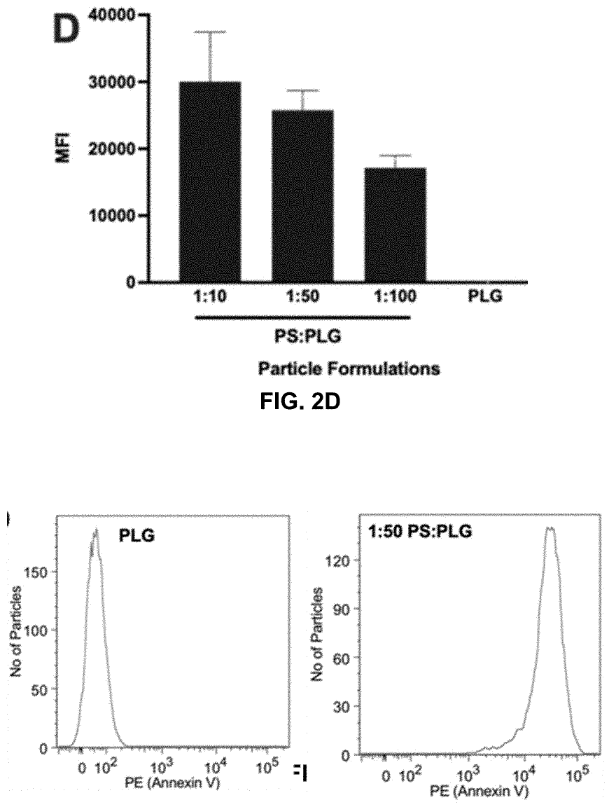

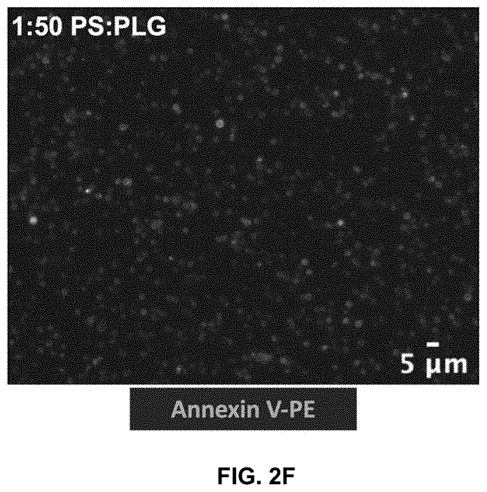

For particle experiments, seeding density was 25,000 cells/cm² for RAW 264.7 macrophages and 50,000 cells/cm² for BMDMs. To favor uniform particle dosage throughout the study, particles were administered based on the number of cells seeded. To achieve this, particle volume was calculated based on a sphere with a diameter determined from particle sizing data (FIG. 2B). Particle density was estimated to be 1.34 g/mL, a commonly reported value for 50:50 PLG33. Together these values were used to obtain the number of particles per mass. Herein, particle dosages ranged from 1 to 25 particles per cell seeded.

Impact of particle treatment on cell viability was analyzed using a cell counting kit 8 (CCK8) assay. RAW 264.7 macrophages were seeded in 96-well culture plates at a density of 25,000 cells/cm². 24 hrs after seeding, cells were treated in triplicates with either PLG or PS:PLG particles. After 24 hrs of particle treatment, unbound particles were washed away. Then, 10 μL of CCK8 solution was added to each well and incubated for 4 hrs. Absorbance was measured at 450 nm.

To assess the impact of particle treatment on cell number, RAW 264.7 macrophages were seeded in 12-well culture plates at a density of 25,000 cells/cm². 24 hrs after seeding, cells were treated in triplicates with PLG or PS:PLG particles. After 24 hrs of particle treatment, unbound particles were washed away and cells were removed from culture using trypsin with 0.25% EDTA for 5 minutes at 37° C. Cells were then counted using a hemocytometer.

Propidium iodide (PI) was used to visually assess cell viability after particle treatment. RAW 264.7 macrophages were seeded in 24-well culture plates at a density of 25,000 cells/cm². 24 hrs after seeding, cells were treated in triplicates with 1:50 PS:PLG particles at a dose of 25 particles per cell seeded. After 24 hrs, unbound particles were washed away and cells were incubated with 5 μL of PI solution for 15 mins in the dark. Afterwards, cells were washed and imaged using an EVOS® FL inverted microscope. As a positive control, certain wells were treated with 10% formalin for 30 mins before incubation with PI; these cells did not receive particle treatment.

Cells were seeded in 24-well tissue culture plates and treated in triplicates with PLG-C6 or PS:PLG-C6 particles at varying particle doses. After 6 hrs of particle treatment, cells were washed 6 times to remove unbound particles. Cells were then fixed and stained with Hoechst for 5 mins to delineate macrophage nuclei. Afterwards, cells were visualized using an EVOS® FL microscope. Five representative images were taken of each well. Using ImageJ software, the fluorescent signal from the bound C6 particles was quantified for each image.

BMDMs were plated in 6-well plates at a density of 50,000 cells/cm² for 48 hrs prior to treatment. Cells were treated with either PS:PLG-C6 or PLG-C6 particles. Cells were incubated with particles for 6 hrs at a concentration of 10 particles per cell seeded. Cells were washed three times with PBS to remove non-associated particles. To assess particle interaction, cells were collected by adding 348 μL of Trypsin with 0.25% EDTA for 5 mins at 37° C. Trypsin was diluted with 616 μL of complete media (DMEM/F12 supplemented with 10% FBS, and 1% penicillin-streptomycin) and cells were scraped with a rubber policeman and transferred to a 5 mL conical tube. Cells were spun down at 300×g and 4° C. for 5 mins with supernatant decanted. Cells were incubated in 0.5 μg of Trustain fcX (anti-CD16/32) diluted in 50 μL of MACS buffer (PBS, 0.5 mM EDTA, 30% BSA) for 15 mins on ice prior to the addition of 0.125 μg of APC anti-mouse F4/80 antibody in 100 μL of MACS buffer for 30 mins on ice. Cells were washed with MACS buffer to remove unbound antibody and then fixed with 0.5 mL of fixation buffer (4% paraformaldehyde in 1×PBS solution), followed by a second wash and filtration through a flow tube filter top. A FACS Aria flow cytometer (BD Biosciences) was used for data acquisition. Fluorescence minus one (FMO) controls were used to set gates for F4/80+ cells and cells associated with C6-labeled particles. Flow cytometry data was acquired using BD FACSDiva software and analyzed with FlowJo.

BMDMs were seeded on coverslips for confocal imaging. Coverslips were first washed under running deionized water, followed by regular dips into 95% ethanol and acetone twice. Coverslips were allowed to dry and sterilized in dry heat oven at 200° C. for 4 hrs. To ensure cell adherence, coverslips were placed into wells of a 6-well plate and coated by incubating coverslips with 50 μg/mL of collagen diluted in sterile PBS overnight at 4° C. Afterwards, wells containing the coverslips were washed 3 times with sterile PBS and seeded with BMDMs at 50,000 cells/cm² for 24 hrs prior to particle treatment for 6 hrs. After particle treatment, cells were washed 3 times with 37° C. sterile PBS to remove particles that were not internalized. Cells were fixed with 2% PFA for 10 mins and rinsed with sterile PBS twice for 15 mins. Cells were stained with F4/80-APC diluted in 1% BSA/PBS, at 37° C. for 1 hr and then rinsed with sterile PBS twice for 15 mins. Finally, cells were stained with DAPI diluted in PBS for 20 mins, mounted with DABCO and then imaged using a Zeiss LSM 510 META Confocal Scanning Laser Microscope. Images at a point between the apical and basal levels of the cell monolayer was obtained.

BMDMs were plated in 6-well plates at a density of 50,000 cells/cm² for 48 hrs prior to treatment. Cells were treated with either PS:PLG-C6 or PLG-C6 particles at a concentration of 10 particles per cell seeded. After each particle treatment, merged GFP and phase contrast images were taken using a 20× objective every 5 secs for 1 hr, to observe live particle internalization by the cells. During this time period, videos of particle internalization was obtained using the time lapse option on the Invitrogen EVOS® FL Auto 2.0 Imaging System with onstage incubator. The onstage incubator was set to 37° C. and CO₂ at 5%, O₂ at 12% and humidity at 77% with automatic adjustments done by the system. Videos were made with a framerate of 30 images per second and processed with the EVOS® FL Auto software.

To analyze the frequency of particle internalization per cell, the number of particles in close contact with each cell was manually counted and of these particles, the number of particles observed to be internalized per cell was recorded for the entire length of each video. Average of 30 cells per video was analyzed, with cells having no contact with any particle omitted from analysis.

The impact of particle treatment on TNF-α, IL-10 and TGF-β1 secretion by RAW 264.7 macrophages was investigated. First, macrophages were seeded in 12-well tissue culture plates and then treated with either PLG and PS:PLG particles at a dose of 10 particles per cell seeded. 24 hrs after treatment, conditioned media was extracted, centrifuged and supernatant decanted, leaving residual particles behind. Factors in conditioned media were measured and analyzed using Duoset ELISA kits according to the manufacturer's instructions. In a separate experiment, the impact of particle treatment on LPS-induced TNF-α, IL-10 and TGF-β1 secretion by RAW 264.7 macrophages were investigated. Macrophages were also seeded in 12-well culture plates but this time, co-treated with LPS and either PLG or PS:PLG particles at varying particle doses (5, 10 and 25 particles per cell seeded).

To measure secreted factors from particle-treated primary macrophages. BMDMs were seeded in 6-well tissue culture plates. Particle treatment and conditioned media extraction were done as described in the paragraph above.

GraphPad Prism software (San Diego, CA) was employed for all statistical analysis. Data are presented as mean±standard deviation, with at least 3 replicates per group unless otherwise stated. Error bars represent standard deviation of the means. Where appropriate, comparisons between three groups at a single time point were made with a one-way ANOVA followed by Tukey's multiple comparisons test, comparisons between two or more groups over time were conducted with a two-way ANOVA followed by Tukey's multiple comparisons test and the comparisons between two groups at a single time point were made with an unpaired t test. The students unpaired t-test. Asterisks indicate p-values of *<0.05, **<0.01, and ***<0.001 to show significant difference between the groups. Specific details regarding statistical analyses carried out for each data set are described in the figure legend.

### Example 1

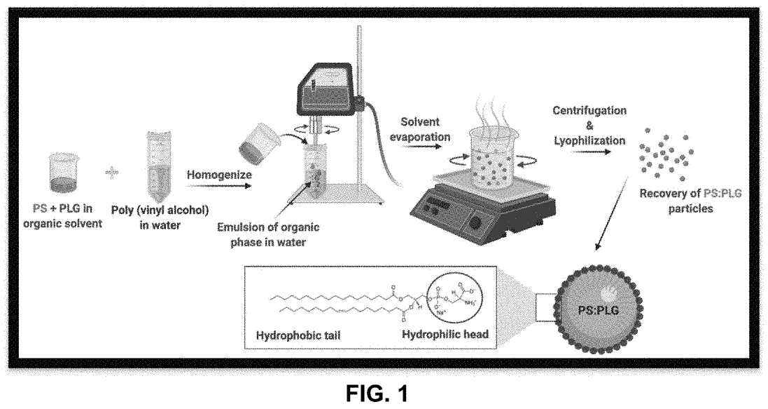

PS:PLG particles were prepared using a single oil-in-water emulsification/solvent extraction method (FIG. 1). PS and PLG were co-dissolved in DCM to obtain the organic phase. Four mass ratios were produced, 1:10, 1:50, 1:100, and 1:200 (PS:PLG). In addition, PLG particles were produced in the absence of PS. For each of these formulations, PLG was maintained at 4% (wt/wt) in DCM and the mass of PS was changed accordingly. The organic solution (0.6 mL) was added to 4 mL of an aqueous solution of 1% (wt/v) PVA. The solutions were then homogenized at 11000 rpm for 5 mins using a Kinematica PT3100D homogenizer. Solvent evaporation and particle formation was achieved by adding the emulsion to 80 mL of water, which was stirred for 1 hour. The contents of the extraction bath were then passed through a 40 μm filter. Particles were collected via centrifugation (250×g for 10 mins) and washed in ultrapure water three times using the same centrifugation protocol. Washed particles were frozen at −20° C. and then lyophilized overnight. Particles were stored under vacuum in a dry environment at room temperature.

Light microscopy indicated that particles were spherical and the addition of PS to the emulsion did not impact this morphology (FIG. 2A). Particle size was determined from light microscopy images. We found there was a dose dependent decrease in particle size with increasing mass of PS in the emulsion; meanwhile, the CV simultaneously increased. In addition, there was a dose dependent decrease in particle mass yield with increasing mass of PS in the emulsion (FIG. 2B).

PLG:PS particles were incubated with Annexin V-PE, a protein that binds PS, to determine if PS integrates with the PLG particle surface while remaining functional. PS:PLG particles bound to Annexin V-PE were visualized with fluorescent microscopy (FIG. 2C) and quantified with flow cytometry (FIG. 2D). Both techniques indicated there was a dose dependent decrease in Annexin V binding as the mass of PS added to the emulsion decreased. Flow cytometry further indicated that the capacity for Annexin V-PE binding was unimodal for each particle formulation. Representative histograms for 1:50 PS:PLG and PLG particles are shown in FIG. 2E. Annexin V-PE binding to PLG particles was not detected with either microcopy or flow cytometry.

1:50 PS:PLG particles were incubated with DMEM supplemented with 10% FBS for 48 hours at 37° C. to determine the stability of PS on the surface of the PS:PLG particles. Fluorescence microscopy indicated the particles could still bind Annexin V suggesting the continued presence of functional PS on the particle surface (FIG. 2F).

### Example 2

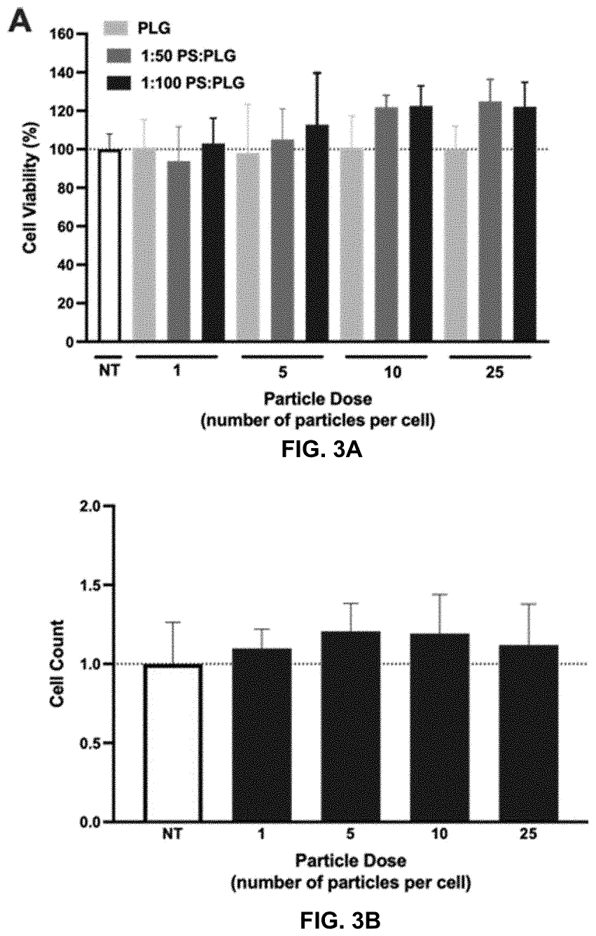

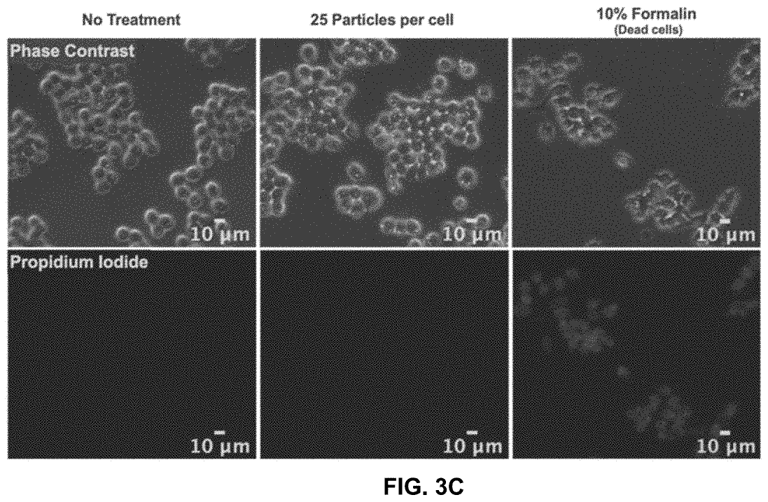

The CCK8 assay was utilized to evaluate the impact of varying dosages of 1:50 and 1:100 PS:PLG particles on RAW 264.7 macrophage viability. After 24 hours, RAW 264.7 macrophage viability did not differ significantly compared to untreated cells (NT) or those treated with PLG particles (FIG. 3A). However, there was a trend for increased formazan production in cultures treated with the PS:PLG particles at the highest particle dosage. In the CCK8 assay, formazan production can be influenced by cell number or cell metabolism (e.g., dehydrogenase activity). Cell number was measured utilizing a hemocytometer after treating RAW 264.7 macrophages with 1:50 PS:PLG particles at varying particle doses for 24 hours. PS:PLG particles treatment did not impact cell number (FIG. 3B).

To assess whether PS:PLG particle treatment impacted plasma membrane integrity (e.g., often used as measure of cell viability), RAW 264.7 macrophages were treated with 1:50 PS:PLG particles at a dosage of 25 particles per cell for 24 hours prior to incubation with propidium iodide (PI). Fluorescent imaging indicated that PS:PLG particles did not impact membrane integrity (FIG. 3C). As a positive control, RAW 264.7 macrophages were treated with 10% formalin prior to the addition of propidium iodide; these cells did not receive particle treatment. Cells treated with formalin exhibited a PI signal, indicating the assay was functioning properly.

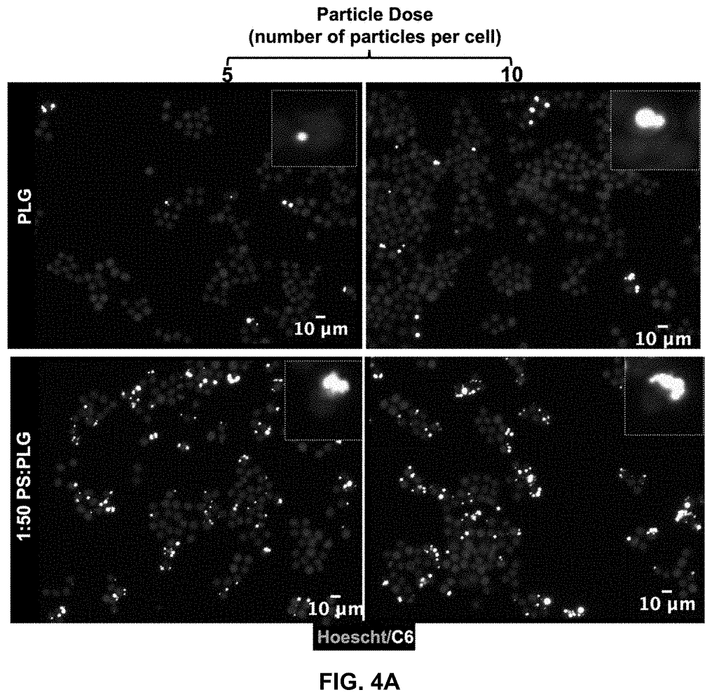

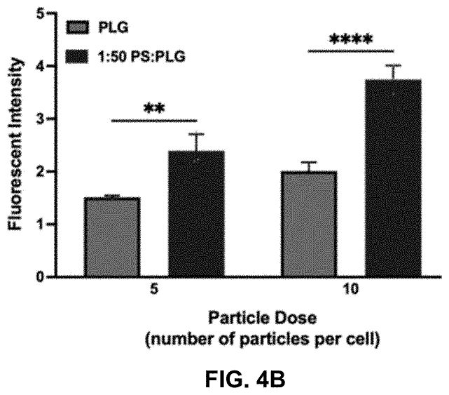

To assess if PS could improve particle interaction with macrophages, RAW 264.7 macrophages were treated with PLG or 1:50 PS:PLG particles loaded with the fluorophore, coumarin 6 (C6). To do so, 0.3 mg/mL C6 was dissolved in DCM along with PLG particles alone or in combination with PS particles. These particles are referred to as PLG-C6 or 1:50 PS:PLG-C6, respectively. RAW 264.7 macrophages were treated with said particles for 6 hours and then unbound particles were washed away. Cells were then fixed, counterstained with Hoechst, and imaged. Fluorescent imaging indicated higher levels of interaction of the 1:50 PS:PLG-C6 particles compared to the PLG-C6 particles for the RAW 264.7 macrophages (FIG. 4A). Analysis of cell-associated C6 particles, using Image) software shows a higher fluorescent intensity for the 1:50 PS:PLG-C6 microparticles compared to PLG-C6 microparticles (FIG. 4B). Thus indicating the PS:PLG microparticles could interact more with macrophages compared to PLG microparticles.

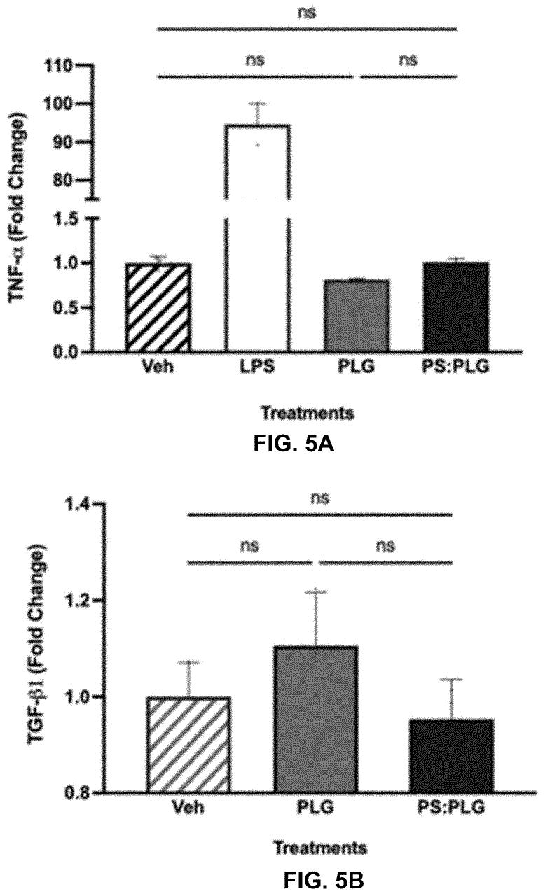

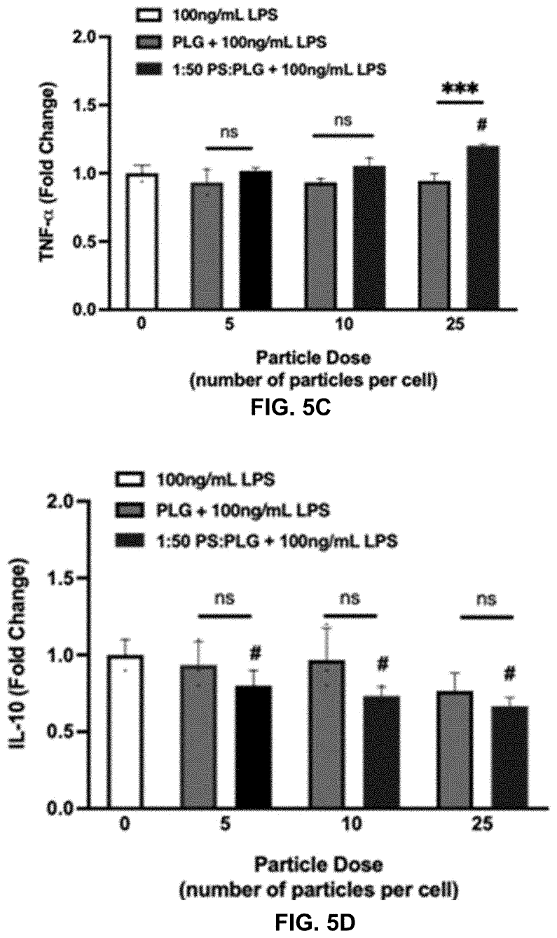

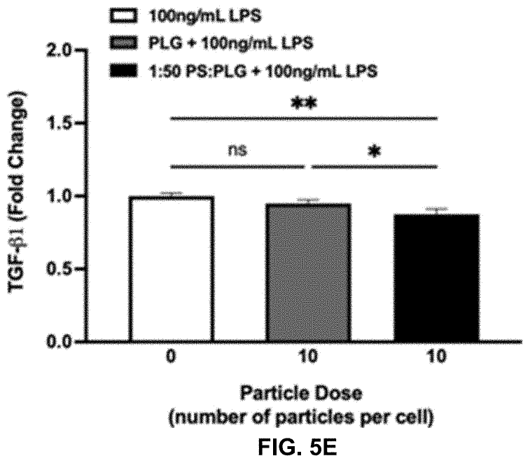

The recognition of PS by macrophages and subsequent engulfment of the expressing apoptotic cells induces a tolerogenic and anti-inflammatory response with modulation of key factors, such as IL-10, TGF-β1 and TNF-α. Thus, macrophage immunomodulatory response to PS:PLG particles was investigated. First, RAW 264.7 macrophages were treated with either PLG or 1:50 PS:PLG particles at a dosage of 10 particles per cell seeded. Secretion of TNF-α, TGF-β1, and IL-10 into the media was then measured after 24 hrs. Neither PLG nor 1:50 PS:PLG particles had a significant impact on TNF-α or TGF-β1 secreted by the RAW 264.7 macrophages (FIG. 5A-5B). LPS control-positive indicated that the cells were capable of TNF-α secretion (FIG. 5A). IL-10 was not detected in the media of resting or particle treated RAW 264.7 macrophages (data not shown).

Next, the effect of PS:PLG on LPS-induced factor secretion by RAW 264.7 macrophages was evaluated. RAW 264.7 macrophages were co-treated with 100 ng/mL of lipopolysaccharide (LPS) and varying dosages of either PLG or 1:50 PS:PLG particles. After 24 hours of treatment, media was collected and subsequently assessed for levels of TNF-α, TGF-β1, and IL-10. Particles at the dosage of 5 and 10 particles per cell had no significant effect on LPS-induced TNF-α secretion. However, at 25 particles per cell dose, PS:PLG particles did modestly, but significantly, increase TNF-α (FIG. 5C). In addition, at every concentration, PS:PLG decreased IL-10 secretion induced by LPS, while there was no significant effect of the PLG particles (FIG. 5D). Finally, TGF-β1 secretion in the presence of LPS was slightly, but significantly decreased by PS:PLG particles at the single dose tested (10 particles per cell) (FIG. 5E).

### Example 3

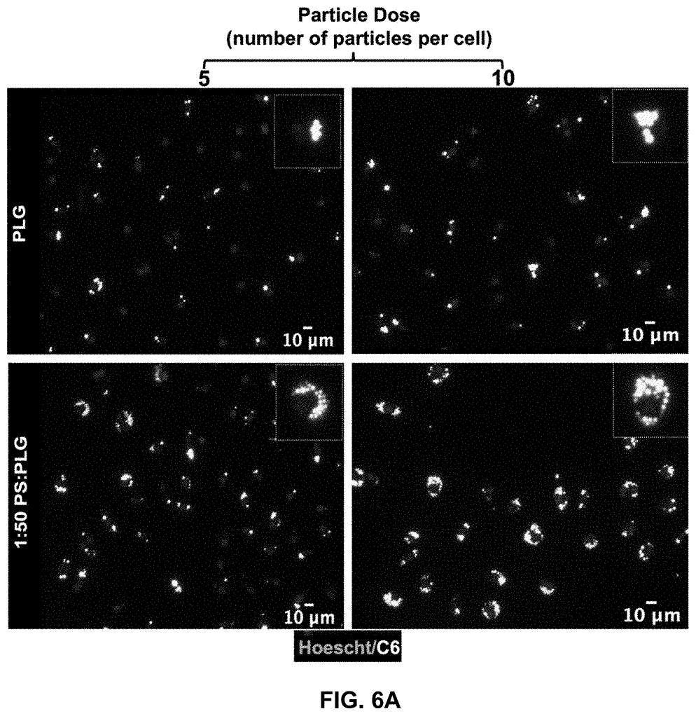

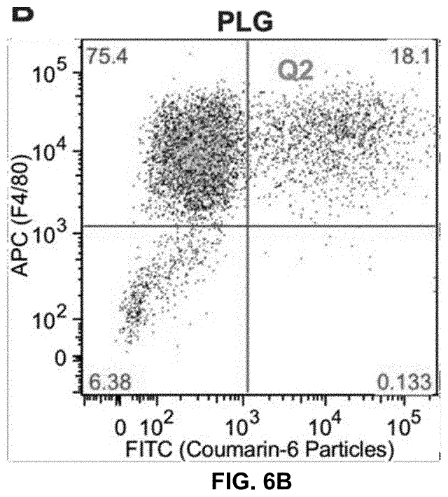

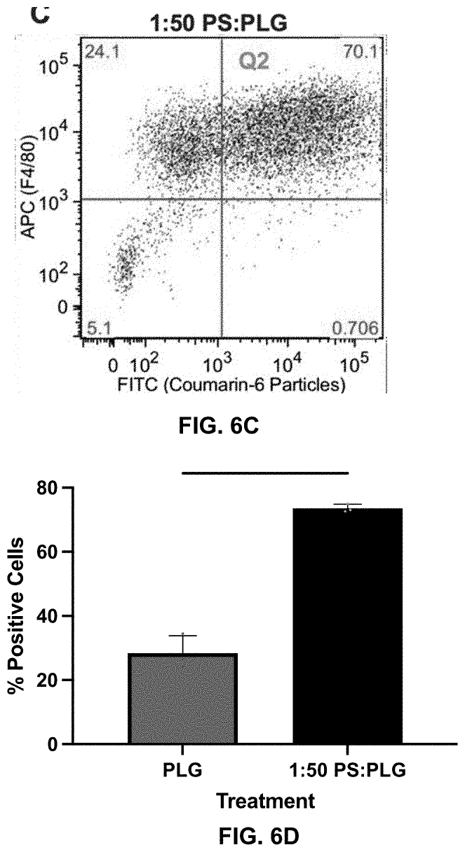

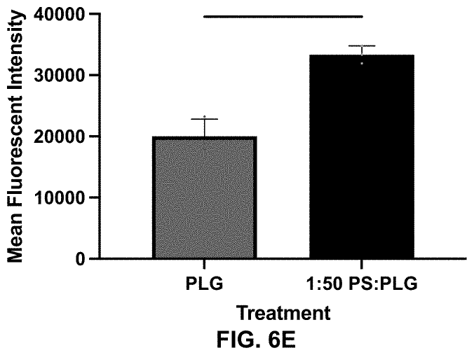

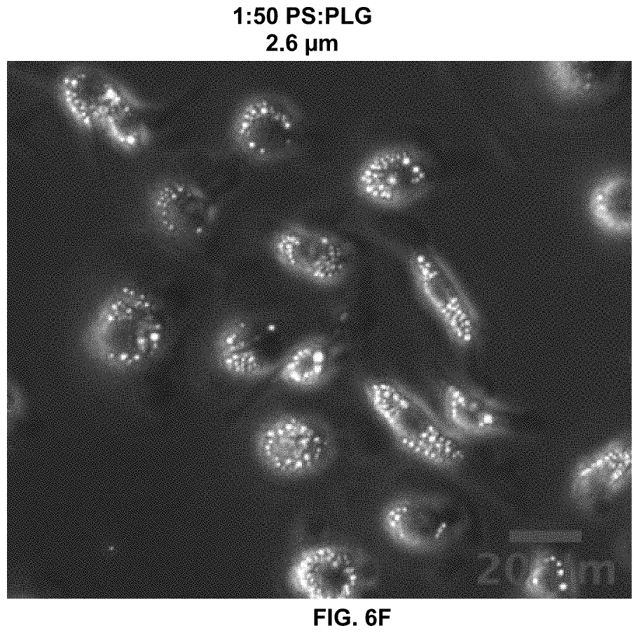

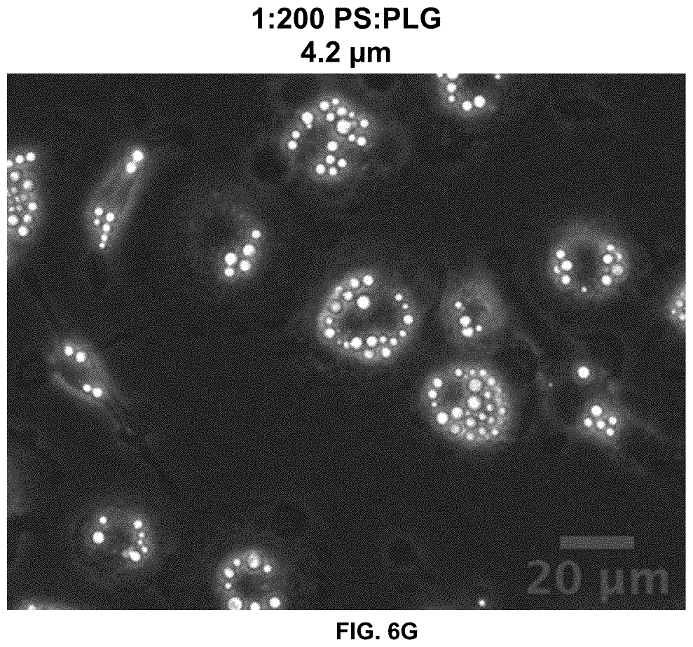

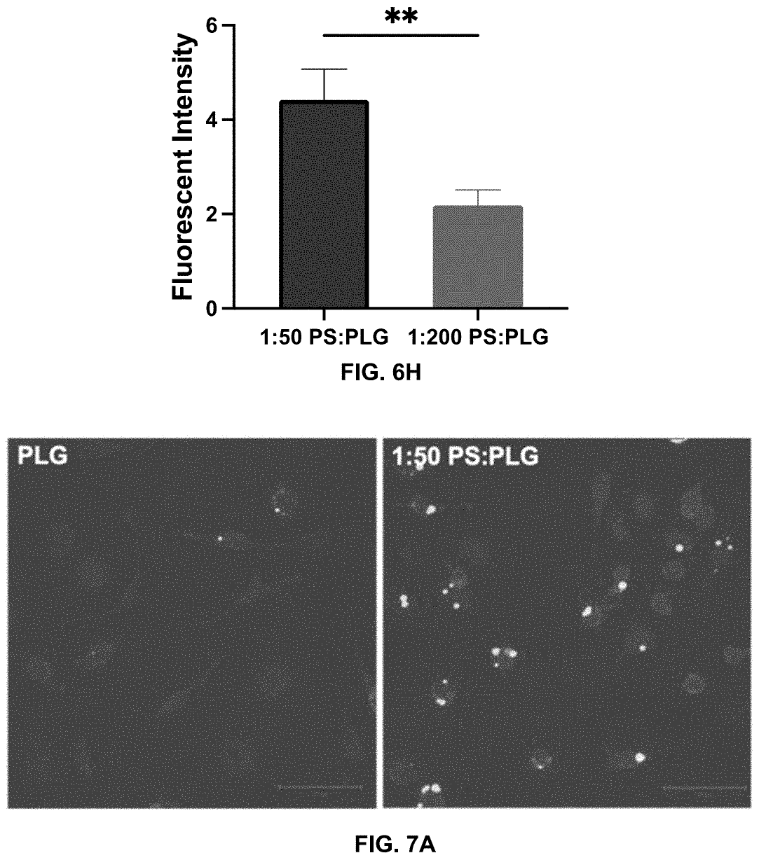

Certain functional differences in phagocytosis and phagocytic pathways have been reported between RAW264.7 macrophages and primary macrophages, such as BMDMs. Thus, presumably prompting differential responses to particle internalization efficiency. Owing to this knowledge, the extent to which PS:PLG particles could interact with BMDMs that were generated from the femur and tibia marrows of female ICR was also evaluated. As expected, fluorescent imaging indicated higher levels of interaction of the 1:50 PS:PLG-C6 particles compared to the PLG-C6 particles (FIG. 6A). Congruent to this visual data, flow cytometric bivariate plots of APC (F4/80) versus FITC (C6-labeled particles) shows a higher percent of particle interaction from macrophages treated with PS:PLG-C6 particles (FIG. 6C) compared to macrophages treated PLG-C6 particles (FIG. 6B) as shown in quadrant 2 (Q2). Quantitatively, the percent of cells positive for PS:PLG-C6 particles is significantly more than cells % for PLG-C6 particles (FIG. 6D). In addition, the median fluorescence intensity of macrophages that were positive for PS:PLG-C6 particles was significantly higher than those positive for PLG-C6 particles, indicating greater particle interaction of PS:PLG particles (FIG. 6E). Additionally, primary macrophages are able to take PS:PLG particles of various sizes including 2.6 micron particles (FIG. 6F) and 4.2 micron (FIG. 6G). Additionally, the extent of particle uptake by macrophages may be tuned by the mass ratio of PS and PLG used during particle fabrication (e.g., 1:50 PS:PLG versus 1:200 PS:PLG) (FIG. 611, fluorescence intensity measures particle uptake because they particles contain the fluorescent dye C6).

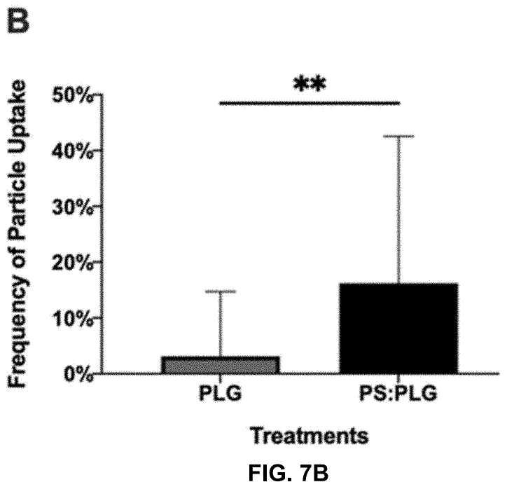

PS ability to improve PS:PLG particle internalization by BMDMs was investigated. To do this, BMDMs seeded on coverslips were treated with C6-labeled particles for 6 hours, stained, and imaged with a confocal laser scanning microscope. Confocal images obtained at a point between the apical and basal levels of the cell monolayer shows green C6 fluorescent signal. Hence, indicating the presence of C6-labeled particles in the cells, as a consequence of particle internalization (FIG. 7A). Even more, the confocal images shows more internalized PS:PLG-C6 particles by the cells compared to particles that had no PS on their surface, PLG-C6 particles. Thus suggesting that PS on the surface of PS:PLG particles facilitates particle internalization by BMDMs.

Live cell imaging of BMDMs treated with C6-particles for 1 hour captures various particles being internalized as they come in close contact with macrophages. Also, not only do the macrophages internalize more PS:PLG-C6 particles more than PLG-C6 particles but the frequency in which PS:PLG-C6 particles are being internalized as they come in close contact with cells is significantly more than the frequency of PLG-C6 particle internalization (FIG. 7B).

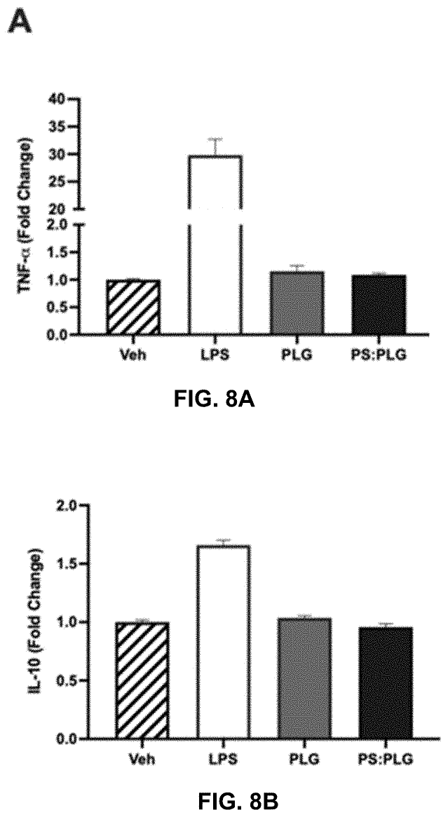

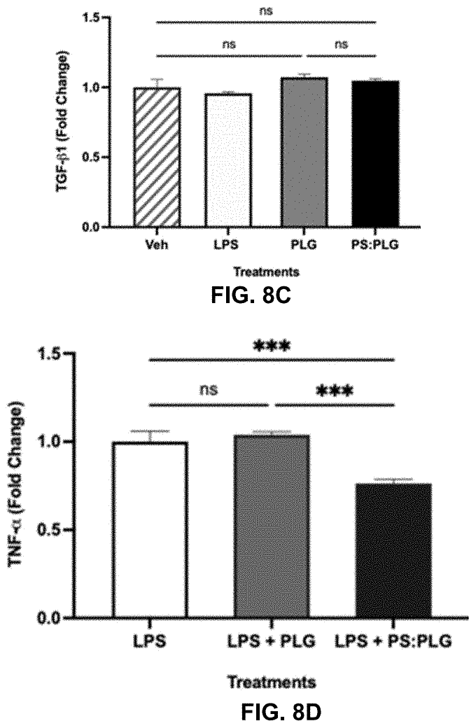

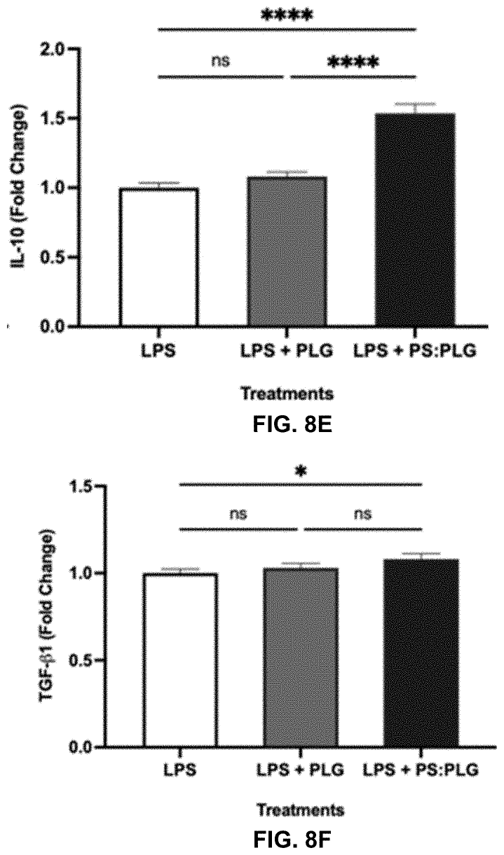

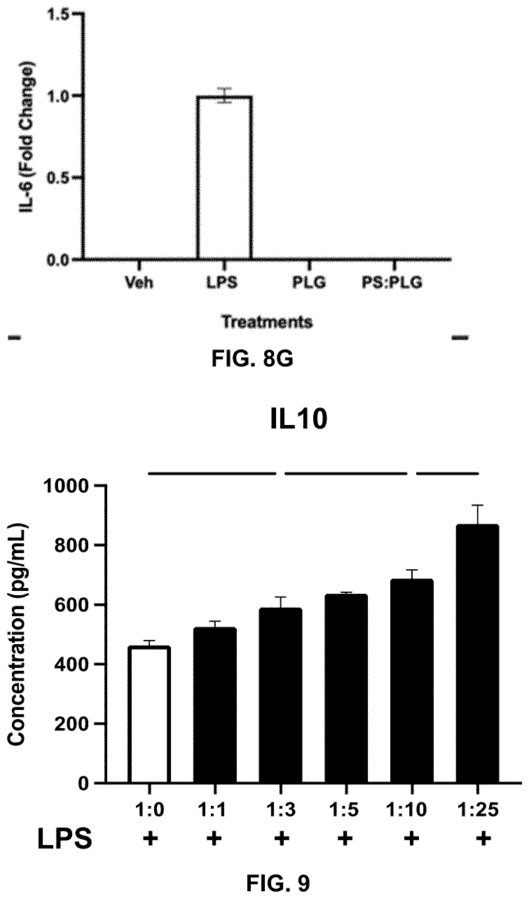

BMDM immunomodulatory response to PS:PLG particles was also investigated. TNF-α, TGF-β1, and IL-10 levels were measured after 24 hours of treating BMDMs with either PLG or PS:PLG particles at a dose of 10 particles per cell seeded. There was no significant impact of PS on both TNF-α and TGF-β1 secreted by BMDMs treated with PS:PLG particles compared to those treated with PLG particles or untreated macrophages (FIGS. 8A & C, compare PS:PLG and PLG to vehicle, Veh). Also, there was no impact IL10 (FIG. 8B). Note that lipopolysaccharide (LPS) is included as a positive control to indicate that these cells can be activated to increase these cytokines (FIG. 8A-C). Also, PS:PLG and PLG particles do not induce IL-6 expression (FIG. 8G). Taken together, the data indicate the particles are not inflammatory in their own right.

The inflammatory response of PS:PLG particles on bone-marrow derived macrophages (BMDMs) upon co-treatment with an inflammatory stimuli, lipopolysaccharide (LPS), was investigated. BMDMs were co-treated with 1 ng/mL of LPS and either PLG or 1:50 PS:PLG particles followed by a measurement of TNF-α, TGF-β1, and IL-10 levels. Compared to other treatment groups, PS:PLG particles significantly decreases LPS-induced TNF-α secretion (FIG. 8D) and also significantly increases IL-10 secretion compared to PLG-treated macrophages or untreated macrophages (FIG. 8E). Indicating that PS could possibly resolve inflammation and promote anti-inflammation from macrophages stimulated with LPS. Furthermore, there was an increase in TGF-β1 secreted by PS:PLG-treated macrophages compared to untreated macrophages but there existed no change when compared to PLG-treated macrophages (FIG. 8F). Also, it was demonstrated that the anti-inflammatory response of PS:PLG on LPS-inflamed macrophages (e.g., increase in IL-10 release) is dose dependent (FIG. 9).

In some embodiments, coumarin 6 (C6) was encapsulated in the PS:PLG and PLG particles. This was achieved by dissolving C6 (0.3 mg/mL) in DCM along with the PS and PLG (or only PLG in some cases). Particle fabrication then proceeded as described above. These particles are referred to as PS:PLG-C6 and PLG-C6, respectively.

Methods disclosed herein are utilized to synthesize 2-3 μm PS-presenting PLG drug delivery particles that significantly promotes particle interaction and internalization by macrophages to effectively deliver small molecules of interest. Surface-functionalizing PLG particles with PS is motivated by the highly conserved process of apoptotic cell recognition through surface PS and subsequent clearance by macrophages. Studies on cancer, obesity, HIV-1, and atherosclerosis have employed PS-modified liposomal-based drug delivery system to enhance macrophage-particle interaction and eventually modulate macrophage function in different cellular microenvironment. However, liposomal particle technology present with certain limitations in liposomal stability, poor drug release profile characterized by an initial burst release and limited control over particle size and shape. Thus, inspiring our exploration of presenting PS on the surface of a different and versatile drug delivery carrier, PLG particles. It is worth noting that previous studies looked at presenting PS in different geometric shapes of PLG nanoparticles using the Particle Replication in Non-Wetting Template (PRINT) fabrication technique. Said studies found that presenting PS on PLG nanoparticles of different shapes promotes immune tolerance through interactions with bone-marrow derived dendritic cells (BMDCs). However, the response of macrophages to PS-presenting PLG particles remains unexplored. Here, methods disclosed herein found that PS can be presented on the surface of micron-sized PLG particles using a very scalable and attractive approach. The single oil-in-water emulsion/solvent extraction technique employed described herein is a batch process which may be utilized to produce up to 109 particles or more in a matter of hours, depending on the size of the vessel being used compared to a template assisted systems that could only produce up to 105 particles per template. In addition, fabricating PLG particles using the emulsion/solvent extraction technique, presents tunable parameters in engineering both particle size, shape, morphology, surface chemistry and encapsulating both hydrophobic and hydrophilic drugs with controlled release rates.

Although, there was a slight decrease in the average size of PS:PLG particles with an increase in the amount of PS added to the emulsion during fabrication but the PS:PLG particle formulations were all within our desired average size range of 2-3 μm. Such decrease in the average size of the PS:PLG particles could be as a result of the physicochemical characteristics of PS. PS is an amphiphilic phospholipid which may make it act as an emulsifier in our fabrication system. Emulsifiers reduce the interfacial tension between the organic phase (DCM and PLG) and aqueous phase (water and PVA), which favors formation of smaller droplets during emulsification and thus, formation of smaller particles. Meanwhile, the addition of PS to the emulsion during fabrication also led to subtle increases in particle size CV which is a measure of particle size variability, with the 1:10 PS:PLG particles having the highest size variation thus suggesting higher size variabilities with the addition of PS. It is possible that such decrease in particle size influenced mass yield. Differential centrifugation conditions in our fabrication system was optimized to recover particles within a size range of 2-3 μm, while leaving smaller particles in suspension. We suspect that the tendency of PS to decrease particle size led to a substantial number of particles with sizes below 2-3 hence explaining a subsequent decrease in mass yield with an increase in the amount of PS added to the emulsion. On the whole, the addition of PS to leading to a dose dependent decrease in particle size and mass yield and subtle increases in particle size CV, suggested that PS was being incorporated into the particles to have such effects; however, to confirm if functional PS was indeed on the particle surface, particles were incubated with annexin V, a protein that binds PS on the surface of apoptotic cells. Annexin V already came labeled with a fluorophore, phycoerythrin (PE, red). When visualized under the same conditions, images of (PS:PLG) particles showed a red fluorescent signal together with an absence of signal for particles that do not contain PS (PLG particles). This suggests that at least some of the PS is present on the surface of the particles. As expected, median fluorescent intensity (MFI) of Annexin V-PE decreased with a decrease in mass of PS added to the emulsion during particle synthesis from 30000 to 17000, with no PE signal detected from PLG particles, thus showing a correlation in the mass of PS added to the emulsion during fabrication to the amount of PS on the surface of the particles able to bind annexin V. We chose to focus on the 1:50 PS:PLG and 1:100 PS:PLG particles for in-vitro studies.

PS on the surface of apoptotic, functions as an “eat me” signal to macrophages, thus attracting macrophages to bind and facilitate subsequent engulfment of the expressing dying cell. In vitro studies using RAW 264.7 macrophages and C6-labeled particles reveal that PS:PLG particles significantly interacted more with macrophages with a 2-fold increase in fluorescence intensity compared to particles that had no surface PS, PLG particles. Cell viability studies showed that such increased interaction of the PS:PLG particles with macrophages was well tolerated. A quantitative and visual evaluation of RAW 264.7 macrophage viability after PS:PLG treatment revealed that macrophages remained viable and presumably seemed to increase in number after treatment with varying doses of the 1:50 or 1:100 PS:PLG particles. Thus indicating that the PS:PLG particles are biocompatible and could continually be used for further studies.

In agreement with the particle interaction data obtained using RAW 264.7 macrophages, flow cytometric analysis on primary BMDMs also showed that both the percentage of macrophages that interacted with PS:PLG particles and the amount of PS:PLG particles interacting with each macrophages are approximately 3- and 2-fold higher than the interaction observed from PLG particles respectively. We needed to know for certainty if such interaction translates to particle internalization. To achieve this, representative confocal images of macrophages treated with PS:PLG particles was obtained. At a point between the apical and basal levels of the cell monolayer, we see C6-labeled PS:PLG particles situated close to the nuclei within macrophages thus indicating that the particles are neither above or below the cell monolayer but inside the macrophages. This observation is further strengthened by a time-lapse video which shows particles subsequently being internalized as they come in close contact with macrophages. Hence, suggesting that the PS on the surface of the PS:PLG particles could presumably facilitate particle internalization just like an apoptotic cell. These PS:PLG particles could be employed in existing or future drug formulations to improve the delivery of drug payloads to modulate macrophage response since modulating cellular fate is contingent on a significant particle-cell interaction. Congruent with findings disclosed herein, studies have also shown improved particle internalization of PS-presenting liposomes by macrophages. The key difference between these studies and ours is the particle delivery system and particle size employed. Particles formed by methods disclosed herein are made from PLG and are within 2-3 μm while particles from previous studies are made from liposomes and are submicron. Particles between 2-3 μm are easily internalized by macrophages but evades internalization by off-target cells thus presenting a way to avoid off-target effects. The ability to achieve similar macrophage-targeting capabilities using PLG particle delivery carriers presents a promising therapeutic strategy that can be applied in modifying or improving future PLG drug release formulations or existing FDA-approved drug release formulations.

Macrophages are essential in host immune response. A function which is imperative for effective response to disease states. Interestingly, PS is reported to play a role in mediating the tolerogenic response borne from apoptotic cell clearance by macrophages with the modulation of key factors such as IL-10, TNF-α, and TGF-β. Owing to this knowledge, we investigated macrophage immunomodulatory response to PS:PLG particles. On resting BMDMs, our PS:PLG particles had no significant effect on the level of TNF-α and TGF-β1 secreted but was shown to reduce the level of IL-10 secreted compared to cells treated with PLG particles. On the other hand, PS:PLG particles mediated an anti-inflammatory effect on LPS-stimulated BMDMs, characterized by a significant reduction in TNF-α, and a significant increase in IL-10 but no change in TGF-β1 secreted. Perhaps, significant reduction in TNF-α secretion after treatment with PS-presenting liposomes and the latter reporting a significant increase in IL-10 secretion and a slight but not significant increase in TGF-β1. On RAW 264.7 macrophages, we report a different inflammatory effect of PS:PLG particles. PS:PLG particles mediated a reduction of TGF-β1 by LPS-stimulated RAW 264.7 macrophages and no significant change in both TNF-α and IL-10 secretion at our standard dose of 10 particles per cell seeded. Interestingly, a previous study reveal that PS-presenting liposomes reduces TNF-α secretion by RAW 264.7 macrophages stimulated with LPS. A likely explanation for this difference is that there exists a possibility that RAW 264.7 macrophages respond differently to nanosized liposomal particles used in the study compared to PLG particles of sizes between 2-3 μm used herein. It was observed that, if treated at a higher dose of 25 particles per cell seeded, TNF-α secretion increases and IL-10 decreases. Owing to this observation, a possible explanation for the discrepancy in the response of LPS-stimulated BMDMs and RAW264.7 macrophage to our PS:PLG particles could be because of the differential regulation of several inflammatory genes when BMDMs and RAW 264.7 macrophages are treated with LPS.

In all, the delivery of drugs or small molecules with particle drug delivery carriers that promotes particle interaction and particle internalization by macrophages might reduce side effects and improve drug efficacy by several folds. Hence, without wishing to be bound by theory, it is believed that these PS-presenting PLG drug delivery carriers may have promising therapeutic implications when conveying small molecules to macrophages in tissue regeneration studies and in targeting macrophages with therapeutic small molecules in several other disease states. Even more, this strategy can be applied to optimize existing PLG drug formulations that target macrophages, especially those that are FDA approved. Future studies aim to test these PS:PLG particles in an in vivo setting. This information will further inform development of optimized particle formulations best suited to be delivered in vivo.

### Example 4

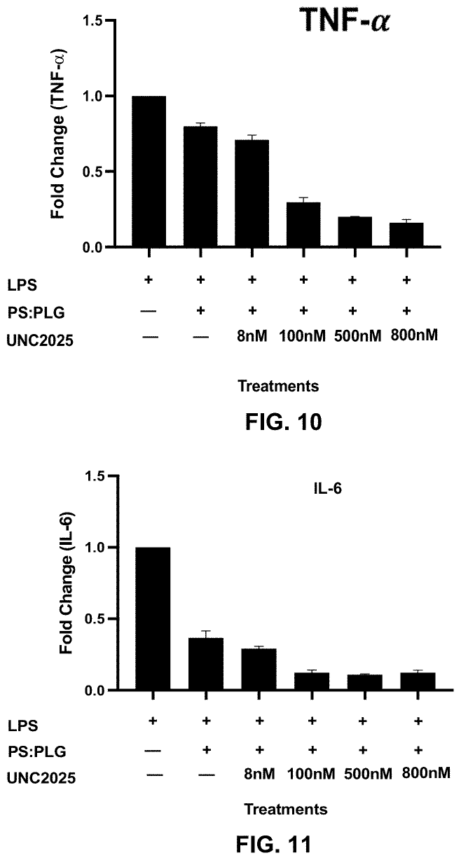

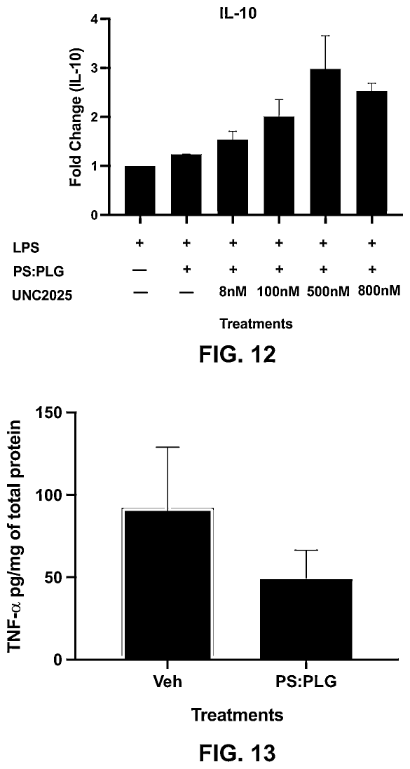

It is demonstrated that the anti-inflammatory response of PS:PLG particle treatment of LPS-inflamed primary macrophages can be synergistically enhanced with co-delivery of small molecules such as UNC2025 (FIGS. 10-12). This effect is not limited to UNC2025 but applicable to a broad range of bioactive molecules including small molecules, nucleic acids, peptides, lipids, glycans, etcetera.

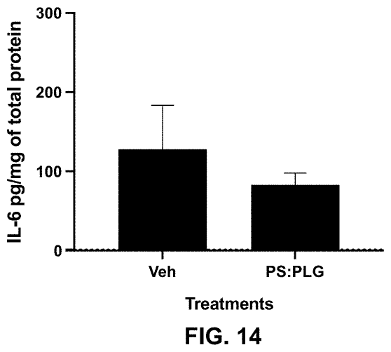

It is demonstrated that intraperitoneal injection of PS:PLG particles into obese mice with inflamed fat tissue leads to decrease in inflammatory cytokine production within visceral fat tissues (FIGS. 13 and 14). In this experiment C57BL/6J mice fed a high fat diet for 16 weeks were injected with 1 mg of PS:PLG particles or vehicle (sterile saline for injection). 24 hours later, mice were euthanized and epididymal fat pads were dissected, homogenized, and assayed for cytokines using ELISA. Thus, PS:PLG particle delivery can modulate inflammation in inflamed fat tissue.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. A method for forming a polymeric particle comprising:
combining an aqueous phase with an organic phase to form an emulsion, the aqueous phase comprising a first emulsifier, the organic phase comprising a second emulsifier and a biocompatible polymer dissolved in a solvent, the emulsion comprising droplets of the organic phase dispersed in the aqueous phase; and
removing at least a portion of the solvent from the organic phase upon which the biocompatible polymer solidifies to form a polymeric particle, wherein the second emulsifier is present at a surface of the solidified polymeric particle.

2. The method of claim 1, wherein the weight ratio of the second emulsifier to the biocompatible polymer is from about 1:5 to about 1:500.

3. The method of claim 1, wherein the weight ratio of the second emulsifier to the biocompatible polymer is from about 1:10 to about 1:200.

4. The method of claim 1, wherein the first emulsifier comprises polyvinyl alcohol, hydroxyethyl cellulose, carboxymethyl cellulose, methyl cellulose, gelatin, alkylarylsulfonates, alkylsulphates, fatty acid salts of alkali metals, polyethylene glycol, poly(ethylene-alt-maleic acid), didodecyldimethylammonium bromide, or a combination thereof.

5. The method of claim 1, wherein the first emulsifier is present in the aqueous phase in an amount of from about 0.1 wt. % to about 3 wt. %.

6. The method of claim 1, wherein the second emulsifier comprises a functionalized phospholipid.

7. The method of claim 6, wherein the functionalized phospholipid comprises phosphatidylserine, phosphatidylethanolamine, phosphatidylcholine, phosphatidylinositol, or a combination thereof.

8. The method of claim 1, wherein the biocompatible polymer comprises poly(ethylene oxide)-poly(propylene oxide)-poly(ethylene oxide) copolymer, poly(lactide-co-glycolide), polycaprolactone, poly(lactic acid), poly(glycolic acid), polyethylene glycol, polysorbate, or a combination thereof.

9. The method of claim 1, wherein the solvent comprises toluene, xylene, dichloromethane, chloroform, trichloroethylene, tetrachloroethylene, tetrachloroethane, chlorobenzene, dichlorobenzene, ethyl acetate, butyl acetate, ethyl formate, methylethyl ketone, or a combination thereof.

10. The method of claim 1, wherein the solidified polymeric particle size is from about 100 nm to about 20 μm.

11. The method of claim 1, the organic phase further comprising a biologically active agent and/or a detectable label.

12. The method of claim 11, wherein the biologically active agent is selected from a group consisting of small molecules, proteins, carbohydrates, lipids, glycosides, indoles, peptides, polyphenols, nucleic acids, glycans, glycoproteins, glycosaminoglycans, and lipoproteins.

13. The method of claim 11, wherein the biologically active agent is a tyrosine kinase inhibitor.

14. A method for forming a polymeric particle comprising:
combining an aqueous phase with an organic phase to form an emulsion, the aqueous phase comprising a first emulsifier, the organic phase comprising a second emulsifier, a biocompatible polymer, a biologically active agent, and a solvent, the emulsion comprises droplets of the organic phase dispersed in the aqueous phase; and
removing at least a portion of the solvent from the organic phase, upon which the biocompatible polymer solidifies to form a polymeric particle, wherein the second emulsifier is present at a surface of the solidified polymeric particle.

15. The method of claim 14, wherein the weight ratio of the second emulsifier to the biocompatible polymer is from about 1:5 to about 1:500.

16. The method of claim 14, wherein the weight ratio of the second emulsifier to the biocompatible polymer is from about 1:10 to about 1:200.

17. The method of claim 14, wherein the first emulsifier comprises polyvinyl alcohol, hydroxyethyl cellulose, carboxymethyl cellulose, methyl cellulose, gelatin, alkylarylsulfonates, alkylsulphates, fatty acid salts of alkali metals, polyethylene glycol, poly(ethylene-alt-maleic acid), didodecyldimethylammonium bromide, or a combination thereof.

18. The method of claim 14, wherein the first emulsifier is present in the aqueous phase in an amount of from about 0.1 wt. % to about 3 wt. %.

19. The method of claim 14, wherein the second emulsifier comprises a functionalized phospholipid.

20. The method of claim 19, wherein the functionalized phospholipid comprises phosphatidylserine, phosphatidylethanolamine, phosphatidylcholine, phosphatidylinositol, or a combination thereof.

21. The method of claim 14, wherein the biocompatible polymer comprises poly(ethylene oxide)-poly(propylene oxide)-poly(ethylene oxide) copolymer, poly(lactide-co-glycolide), polycaprolactone, poly(lactic acid), poly(glycolic acid), polyethylene glycol, polysorbate, or a combination thereof.

22. The method of claim 14, wherein the solvent comprises toluene, xylene, dichloromethane, chloroform, trichloroethylene, tetrachloroethylene, tetrachloroethane, chlorobenzene, dichlorobenzene, ethyl acetate, butyl acetate, ethyl formate, methylethyl ketone, or a combination thereof.

23. The method of claim 14, wherein the solidified polymeric particle size is from about 100 nm to about 20 μm.

24. The method of claim 14, wherein the biologically active agent comprises an amphiphilic phenolic compound.

25. The method of claim 14, wherein the biologically active agent is selected from a group consisting of small molecules, proteins, carbohydrates, lipids, glycosides, indoles, peptides, polyphenols, nucleic acids, glycans, glycoproteins, glycosaminoglycans, and lipoproteins.

26. The method of claim 14, wherein the biologically active agent is a tyrosine kinase inhibitor.
